(19) United States
(12) Patent Application Publication
(10) Pub. No.: US 2004/0109897 A1
(43) Pub. Date: Jun. 10, 2004

(54) Free flowing solid antimicrobial composition

(76) Inventor: Ioana Annis, Mundelein, IL (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/313,152

(22) Filed: Dec. 6, 2002

(51) Int. Cl. A61K 31/34; A61K 9/14

(52) U.S. Cl. 424/489

(57) ABSTRACT

A solid antimicrobial composition that maintains free flow when exposed to high humidity and elevated temperature. The composition contains at least ortho-phthalic aldehyde and an anti-caking agent. A dispersant and/or a solubility enhancer may also be added. The composition is novel because it presents greatly decreased caking tendency as compared to OPA by itself, and preserves excellent flowing properties even after extended exposure to extreme temperature and humidity conditions.

### BACKGROUND OF THE INVENTION

Aromatic aldehydes, and especially ortho-phthalic aldehyde (OPA), are well known for their excellent bacteriostatic and fungistatic properties. OPA solutions are useful especially in disinfecting and sterilizing medical devices. One of the challenges in the use of OPA is its pronounced caking tendency. Thus, even brief exposure to moisture or elevated temperature leads to the agglomeration and compaction of the OPA to yield hard blocks. This requires extra steps in the preparation of OPA solutions, adds to the processing cost, and in certain applications, precludes the use of OPA.

Caking of free flowing powders is an undesired, yet common phenomenon. It takes place when a low moisture, free flowing powder is first transformed into lumps, then into an agglomerated solid, and ultimately, is compacted into a solid block. The occurrence of caking could be determined by several factors. Temperature, moisture, size, shape, mechanical strength of the particles, and position within the powder (pressure) are the most common ones. Caking can occur as a result of electrostatic attraction between particles, solubilization at the particle surface followed by moisture equilibration and hardening, or inter-particle recrystallization. Flow conditioners, also known as anti-caking agents, glidants, anti-agglomerating agents, or free flowing agents, are inert, finely divided solids that are added to a host powder to improve flowability. In order for a conditioner to be effective, its particles must adhere to the host powder and prevent its particles from interacting.

### SUMMARY OF THE INVENTION

The invention provides a solid antimicrobial and antifungal composition that maintains free flowing characteristics upon storage or exposure to elevated temperatures or humidity levels. The composition comprises at least ortho-phthalic aldehyde and one or more anti-caking agents. A second object of the invention is to provide a process for producing the above antimicrobial and antifungal composition.

### DETAILED DESCRIPTION OF THE INVENTION

The present invention provides an antimicrobial and antifungal solid composition that preserves its free flowing characteristics after exposure to elevated temperature and humidity. The composition contains between 90% and 99.999% OPA and between 0.001% and 10% anti-caking additive. Preferably, it contains between 95% and 99.5% OPA and between 0.5% and 5.0% anti-caking agent.

Anti-caking agents, also called flow agents, as used herein, are defined as any additives, organic or inorganic, that would decrease the caking tendency of a solid composition.

The addition of dispersants and/or solubility enhancers to the proposed composition may further improve its dissolution characteristics. Dispersant is defined as any compound or mixture of compounds that improve the dispersal or diffusion of the composition in a solvent of choice. Dispersants are known to those skilled in the art, and some common examples are: potassium and sodium acetate, poly(ethylene glycol), polyacrylates, starch, cellulose, and crosslinked and swellable polymers. Solubility enhancer is defined as any additive that enhances the solubility of the final composition in a solvent of choice. Non-limiting examples of solubility enhancers are surfactants or other surface-active agents, and water soluble polymers.

The OPA can be prepared by various synthetic procedures. This invention is not limited by the preparation pathway selected for OPA. We recognize that the synthetic pathway, especially the final purification step, can affect the flow and the stability of the final material. Thus, the residue of the solvent used for recrystallization, or for the final rinse, can partially solubilize the particle surfaces, which could lead to particle fusion. The extent of exposure of OPA to moisture or elevated temperatures during the manufacturing process could also affect its hygroscopicity and caking tendency. We believe that the flow agents proposed herein are useful for the flow stabilization of OPA obtained through a variety of synthetic pathways.

In general, there are several mechanisms by which anti-caking agents affect the properties of powders, such as: physical separation of the host particles and inhibition of interparticle interactions; interruption of interparticle liquid bridging; lubrication; competition for water absorption; cancellation of electrostatic forces; and, modification to crystal lattices.

Several compositions of OPA with anti-caking agents have been prepared and found to present improved flow characteristics as compared to OPA. Use of anhydrous and/or hygroscopic inorganic salts, such as magnesium and calcium sulfate, sodium pyrophosphate, sodium carbonate, and sodium trisilicate gave formulations with slightly improved flow over OPA. These additives are expected to reduce the caking tendency by competing with the OPA for the residual moisture and by establishing a physical barrier between the particles. Use of inert powders such as silica and hydrophobically modified silica, resulted in OPA formulations with significantly improved flow at temperatures up to 30° C. and relative humidity of up to 95%. These powders usually function as mechanical barriers between the particles, and also help to absorb and spread any solution phase that may appear at the particle interface. Other inert powders, such as alumina, diatomaceous earth (Celite), magnesium silicate, silicilic acid, sodium trisilicate, and tale, showed a less significant effect. Surfactants, such as sodium dodecyl sulfate were also used, but found to add little benefit. However, use of a surfactant in conjunction with a different anti-caking agent could provide particles with improved caking tendency and better dispersability in solvents.

The most successful anti-caking agents, in our experience with OPA; were those which inhibited the access of moisture by coating the OPA particles with a hydrophobic or partially hydrophobic barrier. Agents such as stearates gave material with longest flow stability under extreme temperature and humidity conditions. By the same mechanism, other fatty acids and fatty acid salts are expected to impart good flow characteristics to the OPA formulation. Thus, compositions of OPA with 1-2% of magnesium or zinc stearate maintained good flow after exposure for two months at 30° C. and 90% RH, and after exposure for one month at 40° C. and 90% RH. Under the same conditions, untreated OPA caked up in one day at 30° C. and 90% RH, and in half a day at 40° C. and 90% RH.

The compositions described here were prepared by dry blending of the ingredients. However, another effective process would be the dissolution of the anti-caking agent in a suitable solvent, and spray coating the OPA with the solution. Preferably, the solvent would have to be a poor solvent for the OPA and easily removable after the spray-coating step. Examples of solvents useful in the coating process are water, alcohols, such as isopropyl alcohol, alkanes, such as pentane and hexanes, and ethers, such as diisopropyl ether.

### EXAMPLES

The following tables and examples summarize the more significant findings. The CABOSIL fumed silica samples were obtained from CABOT. The CABOSIL M5 and EH5 are hydrophilic silica, with exposed hydroxyl groups. The TS series CABOSIL are partially or fully hydrophobically modified silica. OPA was obtained from DSM Fine Chemicals (Austria) or from Sigma Aldrich (Milwaukee, Wis.). All other chemicals used herein were from Sigma Aldrich (Milwaukee, Wis.).

Samples of OPA containing 0.01-0.1% water were ground and mixed with various additives at levels of 0.2% to 5%. The samples were mixed on high-speed rollers for one hour followed by exposure to various temperatures and levels of humidity. The samples were examined at various time intervals for flow properties. Complete caking was defined as all sample stuck together in one solid block. For ease of interpretation and comparison, the following notation was used: 1) Formulations that caked in under two weeks were assigned one star (*); 2). Formulations that caked in two to four weeks were assigned two stars; 3) Formulations that caked in four to eight weeks were assigned three stars; 4) Formulations stable (flowable) over eight weeks were assigned four stars.

Control 1

Ortho-phthalic aldehyde (13.04 g, granular) was placed in a 100 ml jar. The jar was tightly capped and placed on high-speed rollers for one hour. After one hour, the OPA was sticking to the jar walls and had very poor flowability. The lid was removed and the sample was placed in a humidity oven set at 40° C. and 90% RH (relative humidity). After 4 hours of exposure the sample had no flow.

Control 2

Ortho-phthalic aldehyde (10.04 g, granular) was placed in a 100 ml jar. The jar was tightly capped and placed on high-speed rollers for one hour. After one hour, the OPA was sticking to the jar walls and had very poor flowability. The lid was removed and the sample was placed in a humidity oven set at 30° C. and 90% RH (relative humidity). After 24 hours of exposure the sample exhibited no free flow.

Control 3

Ortho-phthalic addehyde (12.04 g, granular) was placed in a 100 ml jar. The jar was tightly capped and placed on high-speed rollers for one hour. After one hour the OPA was sticking to the jar walls and had very poor flowability. The lid was removed and the sample was placed in a humidity oven set at 40° C. and 70% RH (relative humidity). After 4 hours of exposure the sample exhibited no free flow.

### Example 1

Ortho-phthalic aldehyde (13.67 g, granular) and magnesium stearate (0.2940 g) were mixed in a 100 ml jar. The jar was tightly capped and placed on high-speed rollers for one hour. Afterwards the lid was removed and the sample was placed in a humidity oven set at 30° C. and 90% RH (relative humidity). The sample was inspected weekly for flow properties, and after 8 weeks it had preserved its free flow.

### Example 2

Ortho-phthalic aldehyde (13.83 g, granular) and CABOSIL M5 fumed silica (0.2740 g) were mixed in a 100 ml jar. The jar was tightly capped and placed on high-speed rollers for one hour. Afterwards the lid was removed and the sample was-placed in a humidity oven set at 30° C. and 90% RH (relative humidity). The sample was inspected weekly for flow properties, and after 4 weeks it had preserved its free flow.

TABLE I
Testing of free flow preservation of various OPA
formulations at 22.5° C. and 65% RH.

| | | Wt. % of anti- | Caking |
|---|---|---|---|
| # | Anti-Caking agent | caking agent | tendency |
| 1 | Control | 0 | * |
| 2 | HEC QP-09H | 2.4 | ** |
| 3 | Magnesium stearate | 2.5 | **** |
| 4 | Sodium acetate trihydrate | 2.3 | ** |
| 5 | Polyvinyl pyrrolidone | 2.0 | ** |
| 6 | Silicilic acid 100 mesh | 2.7 | *** |
| 7 | Sodium trisilicate hydrate | 2.1 | ** |
| 8 | Sodium pyrophosphate | 2.7 | ** |
| 9 | Sodium carbonate anhydrous | 2.1 | ** |
| 10 | PEG 4000 | 2.5 | ** |
| 11 | Precipitated silica | 2.4 | **** |
| 12 | Alumina 150 mesh | 2.5 | ** |
| 12 | CaSO₄ | 2.5 | ** |
| 14 | Sodium dodecyl sulfate | 1.9 | * |
| 15 | Sodium dodecyl sulfate | 5.0 | * |
| 16 | Talc powder | 2.0 | ** |
| 17 | Zinc stearate | 2.0 | **** |

TABLE II
Testing of free flow preservation of various OPA
formulations at 40° C. and 70% RH.

| | | Wt. % of anti- | Caking |
|---|---|---|---|
| # | Anti-caking agent | caking agent | tendency |
| 1 | Control | 0 | * |
| 2 | CABOSIL M5 | 2.0 | *** |
| 3 | CABOSIL TS 720 | 2.0 | *** |
| 4 | CABOSIL TS 530 | 2.0 | *** |
| 5 | CABOSIL EH5 | 2.0 | *** |
| 6 | CABOSIL TS610 | 2.0 | *** |

TABLE III
Testing of free flow preservation of various OPA
formulations at 40° C. and 90% RH.

| | | Wt. % of anti- | Caking |
|---|---|---|---|
| # | Anti-caking agent | caking agent | tendency |
| 1 | Control | 0 | * |
| 2 | CABOSIL M5 | 2.0 | * |
| 3 | CABOSIL TS720 | 2.0 | * |
| 4 | CABOSIL TS530 | 2.0 | * |
| 5 | CABOSIL TS610 | 2.0 | * |
| 6 | CABOSIL EH5 | 2.0 | * |
| 7 | Magnesium stearate | 2.0 | **^{a} |
| 8 | Magnesium stearate | 5.0 | **^{a} |
| 9 | Zinc stearate | 2.0 | **^{a} |
| 10 | Zinc stearate | 5.0 | **^{a} |
| 11 | Celite 545 | 2.0 | * |
| 12 | MgSO₄ anhydrous | 2.1 | * |
| 13 | Magnesium D-gluconate hydrate | 2.0 | * |
| 14 | Magnesium silicate | 2.0 | * |

^{a}The result was recorded after four weeks, when the formulation was still free flowing.

TABLE IV
Testing of free flow preservation of various OPA
formulations at 30° C. and 90% RH.

| | | Wt. % of anti- | Caking |
|---|---|---|---|
| # | Anti-caking agent | caking agent | tendency |
| 1 | Control | 0 | * |
| 2 | CABOSIL M5 | 2.0 | **^{a} |
| 3 | CABOSIL TS720 | 2.0 | **^{a} |
| 4 | CABOSIL TS530 | 2.0 | **^{a} |
| 5 | CABOSIL TS610 | 2.0 | **^{a} |
| 6 | CABOSIL EH5 | 2.0 | **^{a} |
| 7 | Magnesium stearate | 2.0 | ****^{b} |
| 8 | Magnesium stearate | 5.0 | ****^{b} |
| 9 | Zinc stearate | 2.0 | ****^{b} |
| 10 | Zinc stearate | 5.0 | ****^{b} |

^{a}The result was recorded after four weeks, when the formulation was still free flowing.
^{b}The result was recorded after eight weeks, when the formulation was still free flowing.

What is claimed is:

1. A solid formulation of ortho-phthalic aldehyde mixed with one or more anti-caking agents, wherein the formulation contains from about 90 to 99.999% OPA and from about 0.001% to 10% anti-caking agent.

2. A solid formulation according to claim 1 wherein the formulation contains from about 95 to 99.5% OPA and from about 0.5% to 5% anti-caking agent.

3. A solid formulation according to claim 1 wherein the anti-caking agent is selected from the group consisting of metal stearates, fumed silica, hydrophobically-modified fumed silica, and precipitated silica.

4. A solid formulation according to claim 1 further comprising a dispersant.

5. A solid formulation according to claim 4, wherein the dispersant is selected from the group consisting of potassium acetate, sodium acetate, poly(ethylene glycol), polyacrylates, starch, cellulose, crosslinked polymers and swellable polymers.

6. A solid formulation according to claim 1 further comprising a solubility enhancer.

7. A process for preparing a solid formulation of ortho-phthalic aldehyde mixed with one or more anti-caking agents comprising the steps of:
a) providing ortho-phthalic aldehyde;
b) providing one or more anti-caking agents;
c) dry mixing the ortho-phthalic aldehyde with the anti-caking agent.

8. A process for preparing a solid formulation of ortho-phthalic aldehyde mixed with one or more anti-caking agents comprising the steps of:
a) providing ortho-phthalic aldehyde particles;
b) providing one or more anti-caking agents;
c) dissolving the anti-caking agent in a suitable solvent; and
d) spray-coating the dissolved anti-caking agent onto the ortho-phthalic aldehyde particles.

9. The process of claim 8 wherein the solvent is selected from the group consisting of water, alcohols, alkanes, and ethers.
